(19) United States
(12) Patent Application Publication
(10) Pub. No.: US 2008/0031957 A1
(43) Pub. Date: Feb. 7, 2008

(54) Pulmonary delivery of 1alpha,25-dihydroxyvitamin D3 and co-administration of parathyroid hormone or calcitonin

(76) Inventors: Hector F. DeLuca, Deerfield, WI (US); Margaret Clagett-Dame, Deerfield, WI (US); Lori A. Plum, Arena, WI (US); Moises A. Rivera-Bermudez, Madison, WI (US)

Correspondence Address: QUARLES & BRADY LLP, 33 E. MAIN ST, SUITE 900, P.O. BOX 2113, MADISON, WI, 53701-2113, US

(21) Appl. No.: 11/803,570

(22) Filed: May 15, 2007

Related U.S. Application Data
(60) Provisional application No. 60/800,453, filed on May 15, 2006.

(51) Int. Cl. A61K 31/593; A61K 38/23; A61K 38/29; A61P 3/02

(52) U.S. Cl. 424/489; 514/12; 514/167

(57) ABSTRACT

Pharmaceutical pulmonary compositions, methods of delivery to the lungs of a human and methods of treatment thereof. Pharmaceutical formulations including 1α,25-dihydroxyvitamin D₃, an alcohol such as ethanol, and a polyol such as propylene glycol. Another pharmaceutical pulmonary formulation includes 1α,25-dihydroxyvitamin D₃ and dry bulking powder, which is used in a dry powder inhaler. Another pharmaceutical formulation includes 1α,25-dihydroxyvitamin D₃ and an aerosol propellant, which is used in a metered dose inhaler. The pharmaceutical pulmonary formulations may include a second active pharmaceutical ingredient such as calcitonin or a N-terminal peptide fragment of parathyroid hormone consisting of the first 34 to 38 amino acids of SEQ ID No. 1. Pulmonary delivery of the formulations efficaciously increase serum calcium levels in mammals, manage hypocalcemia, treat calcium metabolic disorder and reduce elevated parathyroid hormone levels.

### CROSS REFERENCE TO RELATED APPLICATIONS

This application claims priority to U.S. patent application Ser. No. 60/800,453 filed on May 15, 2006, which is incorporated herein by reference in its entirety.

### STATEMENT REGARDING GOVERNMENT INTEREST

Not applicable.

### FIELD OF THE INVENTION

The present invention relates to the field of compositions and methods for pulmonary delivery of 1α,25-dihydroxyvitamin D₃ (which is also referred to as ³H-1,25(OH)₂D₃) and co-administration of a parathyroid hormone or calcitonin.

### BACKGROUND OF THE INVENTION

The 1α-hydroxylated metabolites of vitamin D are highly potent regulators of calcium homeostasis in mammals, such as humans. It has also been reported that some of these metabolites have activity in terms of cell differentiation, Ostrem et al., Proc. Natl. Acad. Sci. USA, 84, 2610 (1987).

Examples of such metabolites include 1α,25-dihydroxyvitamin D₃ which is the natural hormone and 1α,25-dihydroxyvitamin D₂ which is the analog in ergosterol series. Structural analogs of these metabolites include compounds having one or more different side chains, different hydroxylation patterns, different stereochemistry, or other suitable differences. Other analogs include 1α-hydroxyvitamin D_{3,}1α-hydroxyvitamin D₂, fluorinated side chain derivatives of 1α,25-dihydroxyvitamin D₃, and homologated side chain analogs. Such compounds exhibit highly potent activity both in vivo and in vitro. Such compounds also possess advantageous therapeutic activity profiles having use in treating a variety of diseases including renal osteodystrophy, vitamin D resistant rickets, osteoporosis, psoriasis and other malignancies.

Therapeutic vitamin D derivatives are conventionally delivered in oral and injectable dosage forms. However, oral dosage forms may be undesirable where a patient has a gastrointestinal disturbance condition such as Crohn's Disease, Inflammatory Bowel Disease, diarrhea or another gastrointestinal disease or condition. Such GI conditions may reduce adsorption of the drug and hinder therapeutic effect. Some patients may also be adverse to oral dosage forms and/or prefer other dosage forms or medical devices.

Oral dosage forms may also be undesirable for administering 1α,25-dihydroxyvitamin D₃ because the compound activates intestinal calcium and phosphorus absorption while being absorbed in the intestine. The 1α,25-dihydroxyvitamin D₃ compound is also susceptible to being degraded by CYP-24 enzymes present in the intestine. Interestingly, metabolism of ingested 1α,25-dihydroxyvitamin D₃ induces CYP-24 enzymes. CYP-24 enzymes degrade 1α,25-dihydroxyvitamin D₃ to a C-23 carboxylic acid (also referred to as calcitroic acid).

Additionally, transdermal dosage forms may not be well-suited for administering vitamin D analogs because, among other reasons, human skin is insufficiently permeable to allow therapeutic dosing. Thus, there exists a need for an effective pharmaceutical dosage form for delivering vitamin D analogs (such as 1α,25-dihydroxyvitamin D₃) to human patients in need thereof. There also exists a need for an effective pharmaceutical dosage form combining vitamin D analogs (such as 1α,25-dihydroxyvitamin D₃) with other active pharmaceutical ingredients to enhance and expand available therapies and indications for human patients in need thereof.

### DESCRIPTION OF DRAWINGS OF EXEMPLARY EMBODIMENTS

FIG. 1 shows the tissue distribution of 1α, 25-dihydroxyvitamin D₃ in Sprague Dawley rats after 10 minutes (n=4) and 4 hours (n=3) after pulmonary delivery of 1α,25-dihydroxyvitamin D₃ in accordance with administering 100 μL of Formulation A, whereby the data shows that at 10 minutes 1α,25-dihydroxyvitamin D₃ was detected primarily in the lungs, trachea and serum, and that at 4 hours 1α,25-dihydroxyvitamin D₃ was detected primarily in the serum and stomach.

FIG. 2 shows the tissue distribution of 1α,25-dihydroxyvitamin D₃ in Sprague Dawley rats after 10 minutes (n=2) and 4 hours (n=3) after pulmonary delivery of 1α,25-dihydroxyvitamin D₃ in accordance with administering 100 μL of Formulation B, whereby the data show that at 10 minutes 1α,25-dihydroxyvitamin D₃ was detected primarily in the serum and secondarily in the trachea and lungs, and that at 4 hours 1α,25-dihydroxyvitamin D₃ was detected primarily in the serum and stomach.

FIG. 3 shows the effect of 1α,25-dihydroxyvitamin D₃ on serum calcium levels in Brown Norway rats in accordance with administering Formulation A (n=6) in a dose containing 1 μg/Kg BW 1α,25-dihydroxyvitamin D₃ (n=8), whereby BW is body weight (or, kg_{BW}).

FIG. 4 shows the effect of 1α,25-dihydroxyvitamin D₃ on serum calcium levels in Brown Norway rats in accordance with administering Formulation A (n=9) in a dose containing 10 μg/kg_{BW} 1α,25-dihydroxyvitamin D₃ (n=9).

### SUMMARY OF THE INVENTION

One aspect of the invention is a method of increasing serum calcium in a human comprising the steps or acts of delivering an atomized dose of a pharmaceutical solution comprising water, one or more alcohols, one or more polyols, and, a first active pharmaceutical ingredient comprising 1α,25-dihydroxyvitamin D₃, or esters or solutes thereof to the lungs of the human.

In an exemplary embodiment of the method, the alcohol is ethanol.

In another exemplary embodiment of the method, the polyol is propylene glycol.

In another exemplary embodiment of the method, the pharmaceutical solution further comprises one or more excipients.

In another exemplary embodiment of the method, the excipient is a nonionic surfactant, sodium chloride, sodium ascorbate, dibasic sodium phosphate, monobasic sodium phosphate, disodium edatate or combinations thereof.

In another exemplary embodiment of the method, the atomized dose of the pharmaceutical solution comprises a dose of the first active pharmaceutical ingredient being 1α,25-dihydroxyvitamin D₃ or esters or solutes thereof in the range of 0.2-10 μg. The dosing regimen for a human is in the range of 0.2-10 μg per day, and more preferably 0.2-2 μg per day. The number of doses administered each day and the amount of 1α,25-dihydroxyvitamin D₃ or esters or solutes thereof in each dose can be varied to achieve the daily dosing regimen.

In another exemplary embodiment of the method, the solution further comprises a second active pharmaceutical ingredient being calcitonin or N-terminal peptide fragment of parathyroid hormone consisting of the first 34 to 38 amino acids of SEQ ID No. 1. As used herein, “calcitonin” refers to synthetic calcitonin, calcitonin-like peptides or calcitonin mimetic, which is described in detail in U.S. patent application Ser. Nos. 10/235,244 filed Sep. 5, 2002, and 11/352,717 filed Feb. 13, 2006, which are incorporated herein by reference in their entirety.

In another exemplary embodiment of the method, the atomized dose of the pharmaceutical solution comprises a dose in the range of 100-2000 μg of the second active pharmaceutical ingredient being the N-terminal peptide fragment of parathyroid hormone consisting of the first 34 to 38 amino acids of SEQ ID No. 1.

In another exemplary embodiment of the method, the atomized dose of the pharmaceutical solution comprises a dose in the range of 100-1000 μg of the second active pharmaceutical ingredient being the calcitonin.

Another aspect of the invention is a pharmaceutical pulmonary composition comprising water, one or more alcohols, one or more polyols, and, a first active pharmaceutical ingredient comprising 1α,25-dihydroxyvitamin D₃ or esters or solutes thereof.

In an exemplary embodiment of the composition, the alcohol is ethanol.

In another exemplary embodiment of the composition, the polyol is propylene glycol.

In another exemplary embodiment of the composition, the composition further comprises one or more excipients.

In another exemplary embodiment of the composition, the excipient is a nonionic surfactant, sodium chloride, sodium ascorbate, dibasic sodium phosphate, monobasic sodium phosphate, disodium edatate or combinations thereof

In another exemplary embodiment of the composition, the composition further comprises a dose of the first active pharmaceutical ingredient being 1α,25-dihydroxyvitamin D₃ or esters or solutes thereof in the range of 0.2-10 μg, and more preferably in the range of 0.2-2 μg.

In another exemplary embodiment of the composition, the composition further comprises a second active pharmaceutical ingredient being calcitonin or an N-terminal peptide fragment of parathyroid hormone consisting of the first 34 to 38 amino acids of SEQ ID No. 1.

In another exemplary embodiment of the composition, the composition comprises a dose of the second active pharmaceutical ingredient being the N-terminal peptide fragment of parathyroid hormone consisting of the first 34 to 38 amino acids of SEQ ID No. 1 in the range of 100-2000 μg/dose.

In another exemplary embodiment of the composition, the composition comprises a dose of the second active pharmaceutical ingredient being the calcitonin in the range of 100-1000 μg/dose.

Another aspect of the invention is a method of increasing serum calcium in a human comprising the steps or acts of delivering a pharmaceutical dose of a first active pharmaceutical ingredient comprising 1α,25-dihydroxyvitamin D₃, or esters or solutes thereof to the lungs of the human.

Another aspect of the invention is a method of managing hypocalcemia in a human undergoing chronic hemodialysis comprising the steps or acts of delivering a pharmaceutical dose of a first pharmaceutical active ingredient comprising 1α,25-dihydroxyvitamin D₃, or esters or solutes thereof to the lungs of the human.

Another aspect of the invention is a method of treating calcium metabolic disorder in a human comprising the steps or acts of delivering a pharmaceutical dose of a first active pharmaceutical ingredient comprising 1α,25-dihydroxyvitamin D₃, or esters or solutes thereof to the lungs of the human.

Another aspect of the invention is a method of reducing elevated parathyroid hormone levels in a human comprising the steps or acts of delivering a pharmaceutical dose of a first active pharmaceutical ingredient comprising 1α,25-dihydroxyvitamin D₃, or esters or solutes thereof to the lungs of the human.

In an exemplary embodiment of any of the above methods, the method further comprises the step or act of co-delivering a pharmaceutical dose of a second active pharmaceutical ingredient selected from the group consisting of calcitonin and an N-terminal peptide fragment of parathyroid hormone consisting of the first 34 to 38 amino acids of SEQ ID No. 1.

In another exemplary embodiment of any of the above methods, the method comprises delivering a dose in the range of 100-2000 μg of the second active pharmaceutical ingredient being the N-terminal peptide fragment of parathyroid hormone consisting of the first 34 to 38 amino acids of SEQ ID No. 1.

In another exemplary embodiment of any of the above methods, the method comprises delivering a dose in the range of 100-1000 μg of the second active pharmaceutical ingredient being the calcitonin.

In another exemplary embodiment of any of the above methods, the method comprises delivering a dose of the first active pharmaceutical ingredient being 1α,25-dihydroxyvitamin D₃ or esters or solutes thereof in the range of 0.2-10 μg, and more preferably in the range of 0.2-2 μg.

Another aspect of the invention is a method of increasing serum calcium in a human comprising the steps or acts of delivering a dose of a pharmaceutical dry powder composition comprising dry bulking powder and a first active pharmaceutical ingredient comprising 1α,25-dihydroxyvitamin D₃, or esters or solutes thereof to the lungs of the human.

Another aspect of the invention is a pharmaceutical pulmonary composition comprising a dry bulking powder, and, a first active pharmaceutical ingredient comprising 1α,25-dihydroxyvitamin D₃, or esters or solutes thereof.

Another aspect of the invention is a method of increasing serum calcium in a human comprising the steps or acts of delivering a dose of a pharmaceutical aerosol composition comprising a propellant and a first active pharmaceutical ingredient comprising 1α,25-dihydroxyvitamin D₃, or esters or solutes thereof to the lungs of the human.

Another aspect of the invention is a pharmaceutical pulmonary composition comprising an aerosol propellant, and, a first active pharmaceutical ingredient comprising 1α,25-dihydroxyvitamin D₃, or esters or solutes thereof.

In an exemplary embodiment of the pharmaceutical pulmonary composition, the composition further comprises a surfactant.

Another aspect of the invention is a dry powder inhaler containing the pharmaceutical pulmonary composition comprising a dry bulking powder, and, a first active pharmaceutical ingredient comprising 1α,25-dihydroxyvitamin D₃, or esters or solutes thereof.

Another aspect of the invention is a metered dose inhaler containing the pharmaceutical pulmonary composition comprising a propellant and a first active pharmaceutical ingredient comprising 1α,25-dihydroxyvitamin D₃, or esters or solutes thereof to the lungs of the human.

### DETAILED DESCRIPTION OF THE EXEMPLARY EMBODIMENTS

The invention relates to pharmaceutical pulmonary compositions, methods of delivery to the lungs of a human and methods of treatment thereof. Pharmaceutical formulations including a first API being 1α,25-dihydroxyvitamin D₃ or an ester or salt thereof, an alcohol such as ethanol, and a polyol such as propylene glycol. Another pharmaceutical pulmonary formulation includes 1α,25-dihydroxyvitamin D₃ and dry bulking powder, which is used in a dry powder inhaler. Another pharmaceutical formulation includes 1α,25-dihydroxyvitamin D₃ and an aerosol propellant, which is used in a metered dose inhaler. The pharmaceutical pulmonary formulations may include a second API such as calcitonin or a N-terminal peptide fragment of parathyroid hormone consisting of the first 34 to 38 amino acids of SEQ ID No. 1. Pulmonary delivery of the formulations efficaciously increase serum calcium levels in mammals, manage hypocalcemia, treat calcium metabolic disorder and reduce elevated parathyroid hormone levels. Pulmonary delivery is also referred to as the route of administration.

As used herein, the term “pharmaceutical” refers to compositions, formulations, solutions, methods, etc. that are suitable and acceptable for pharmaceutical use, whereby all of the components (such as excipients, solvents, additives, surfactants, powder, and the like) are preferably USP grade materials, and whereby the API is (or, APIs are) present in an amount sufficient to impart a therapeutic effect, treatment (prophylactic, treatment of a condition, or the like), and/or benefit to the human.

As used herein, the phrase “pulmonary system” includes the upper respiratory tract, lower respiratory tract, trachea, bronchial tree and lungs, which are commonly understood.

As used herein, “lungs” includes the bronchial tree, respiratory bronchioles, alveolar ducts and alveoli.

Systemic pulmonary delivery of 1α,25-dihydroxyvitamin D₃ is advantageous for several reasons. Systemic pulmonary delivery advantageously delivers the drug directly into the patient's blood, which then circulates the drug throughout the body avoiding breakdown or inactivation in the stomach/gut and/or gastrointestinal tract. Pulmonary delivery also avoids first-pass metabolism in organs such as the liver and kidney. Pulmonary delivery also delivers drug to the patient faster than oral dosage forms. Calcitriol is well known in the art as a compound that stimulates intestinal calcium transport. (Remington: The Science and Practice of Pharmacy, 21st Ed., p. 1698 (2006)). Calcitriol is known to be efficacious for management of hypocalcemia in patients undergoing chronic hemodialysis, for treating calcium metabolic disorder, and for reducing elevated parathyroid hormone levels.

The instant pharmaceutical pulmonary composition may further include a second active pharmaceutical ingredient (“API”) to be co-administered with the 1α,25-dihydroxyvitamin D₃. Suitable second API's include calcitonin and parathyroid hormone (e.g., hPTH).

Calcitonin (also referred to as calcimar and miacalcin) is a 32 amino acid polypeptide hormone secreted by the parafollicular cells of the thyroid gland in mammals. The chemical formula for calcitonin is C₁₄₅H₂₄₀O₄₈S₂ and has a molecular weight of 3431.88. The SEQ. ID. for calcitonin and a description thereof is disclosed in Remington: The Science and Practice of Pharmacy, 21st Ed., p. 1456-1457 (2006), which is incorporated herein by reference.

Calcitonin is known to participate in calcium and phosphorus metabolism. In particular, calcitonin is known to decrease blood calcium levels at least in part by effects on two well-studied target organs. In bone, calcitonin suppresses resorption of bone by inhibiting the activity of osteoclasts releasing calcium and phosphorus into blood. In the kidney, calcium and phosphorus are prevented from being lost in urine by re-absorption in the kidney tubules. Calcitonin inhibits re-absorption of calcium and phosphorus ions leading to increased rates of loss in urine. Calcitonin is known to be efficacious in treating hypercalcemia and Paget disease. Calcitonin is also known to be a valuable aid in managing some forms of osteoporosis.

Human parathyroid hormone (“hPTH”) is a linear polypeptide chain having 84 amino acids. When working properly, hPTH is known to maintain extracellular calcium ions at a constant concentration in the human body. (See Remington at p. 558). It is know that amino acids 1 to 27 of the N-terminal portion of the peptide are associated with biological activity in the human body. At one time, hPTH injections were used extensively on humans to raise plasma calcium levels in hypocalcemic patients. However, hPTH injection is no longer available for clinical use and has been replaced by administration of calcium or vitamin D.

hPTH is also involved in the regulation of phosphorus homeostasis. PTH is also involved in control and regulation of bone growth and bone density. Some N-terminal fragments of hPTH have the same or similar biological activity as the full, intact protein. In particular, N-terminal segments of hPTH comprising amino acids 1-34 (“hPTH34”) and 1-38 (“hPTH38”) are preferred. Native hPTH-(1-84), which contains amino acids 1-84, may also be co-administered as a second API. hPTH(1-84) is a known therapeutic for treating post-menopausal osteoporosis. The hPTH(1-84) may be compound ALX1-11 which is being tested by NPS, Allelix Biopharmaceuticals (Ontario, Canada) and GlaxoSmithKline. Production and delivery of hPTH is known is the art. (See, Morley P, et al, Parathyroid Hormone: An Anabolic Treatment for Osteoporosis, Current Pharmaceutical Design, 2001, Vol. 7, No. 8, p. 671-687, which is incorporated herein by reference). Other various forms of hPTH(1-84) and therapeutic dosing levels thereof are disclosed in U.S. Pat. No. 5,496,801, which is incorporated herein in its entirety by reference. The amino acid sequence for hPTH(1-84) is reported in Kimura et al, Biochem Biophys Res Comm, 114 (2):493, which is also incorporated herein in its entirety by reference.

Recombinantly produced polypeptides having the same sequence of hPTH may also be used. hPTH fragments having carboxyl amino acid extensions beyond the 34 position may also be used. Amino-terminal extensions or α-carboxyl amide substitution at the carboxyl terminus may also be employed. Therapeutically suitable salts and esters of the PTH fragment may also be used.

hPTH34 and hPTH38 each have the amino acid sequence [SEQ. ID No.1] shown in Table 1.

TABLE 1

1 5
H.sub.2 N-Ser-Val-Ser-Glu-Ile-Gln-Leu-Met-His-
10 15
Asn-Leu-Gly-Lys-His-Leu-Asn-Ser-Met-Glu-
20 25
Arg-Val-Glu-Trp-Leu-Arg-Lys-Lys-Leu-Gln-
30 35
Asp-Val-His-Asn-Phe-Val-Ala-Leu-Gly-COOH

hPTH34 and hPTH38 fragments are available commercially from Peninsula Laboratories, Inc., Belmont, Calif.; Sigma Chemical, St. Louis, Mo.; and, Bachem Cali., Torrance, Calif. The PTH fragments may also be produced recombinantly by expression in cultured cells of recombinant DNA molecules encoding the desired fragment of the PTH molecule. Suitable recombinant expression systems and methods are described in the literature. (See, Manniatis, Molecular Cloning: A Laboratory Manual, Cold Spring Harbor, N.Y. 1982). The hPTH34 may also be Eli Lilly's recombinant rhPTH-(1-34) (also referred to as FORTEO™ or Teriparatide). The DNA molecules which are expressed may themselves be synthetic or derived from a natural source. Synthetic polynucleotides may be synthesized by well-known techniques. For example, single-stranded DNA fragments may be prepared by a phosphoraminite method described by Beaucage and Carruthers (1981) Tett. Lett. 22:1859-1862. A double-stranded fragment may then be obtained either by (1) synthesizing the complementary strand and annealing the strands together under appropriate conditions or (2) by adding the complementary strand using DNA polymerase with an appropriate primer sequence. Synthetic DNA sequences can be separated using automated equipment available from Applied Biosystems, Inc., Foster City, Calif.

Pulmonary delivery of molecules containing the PTH34 and PTH38 fragments is disclosed in U.S. Pat. No. 5,814,607, which is hereby incorporated herein by reference in its entirety.

Dry powder inhalation pharmaceutical formulations and devices are known in the art. The first and second API's of the invention may be formulated into a dry powder pharmaceutical formulation for use with a dry powder inhaler (DPI) to administer and deliver the API's within the pulmonary system of a human. The DPI may also be a multi-dose DPI (MDPI or MDDPI). Respirable powders of various particle sizes can be produced using a variety of conventional processes, such as jet-milling, spray drying, solvent precipitation, and the like. The dry powders may then be formulated into a powder mass using dry bulking powders, such as sucrose, lactose, trehalose, human serum albumin, glycine, cellobiose, dextrans, maltotriose, pectin, sodium citrate, sodium ascorbate, marnnitol and the like. The formulated dry powder composition may be packaged in a DPI, such as Aerolizer® available from Novartis Pharma AG and Schering-Plough Corporation, Turbohaler® available from AstraZeneca, Diskus® available from GlaxoSmithKline, Actispire™ available from Brittannia Pharmaceuticals, Twisthaler® available from Schering-Plough, Novolizer® available from Meda Pharma BV, AcuBreathe™ available from Respirics Inc., Certihaler™ available from Skye Pharma, and the like.

Aerosol inhalation pharmaceutical formulations and devices are also known in the art. The first and second API's of the invention may be formulated into an aerosol pharmaceutical formulation for use with a metered dose inhaler (MDI) to administer and deliver the API's within the pulmonary system of a human. The API's may be dissolved or suspended (as a solid) in a pharmaceutically suitable aerosol propellant, such as a hydrofluorocarbon (HFC), preferably a hydrofluoroalkane (HFA). Exemplary HFA's include, but are not limited to, tetrafluoroethane (HFA-134a) and heptafluoropropane (HFA-227). Preferably, the API's are suspended in the aerosol propellant in the form of respirable particles similar to that used in the DPI's described herein. Preferably, the aerosol composition further contains a pharmaceutically suitable surfactant to improve dispersion, such as oleic acid, sorbitan trioleate, and long chain diglycerides or phospholipids. The aerosol composition may further contain a lower alcohol (up to 30 wt %), other additives or excipients to impart chemical stability and physiological acceptability. The aerosol formulation may be packaged in a MDI, which are well known in the art. (See, e.g., Stein SW et al., “Reinventing Metered Dose Inhalers: From Poorly Efficient CFC MDIs to Highly Efficient HFA MDIs,” Drug Delivery Technology 2003, 3:46-51).

### EXAMPLES

Aqueous Formulations.

Sprague Dawley and Brown Norway male rats aged 6-7 weeks were obtained from Harlan Sprague-Dawley (Madison, Wis.) and housed in shoebox cages. Animals were provided a purified rodent diet prepared in-house containing 0.47% calcium and 0.3% phosphorus, and water ad libitum. The diet was supplemented with 1.6 IU vitamin D₃/g diet.

Rats are the preferred species for in vivo analysis of 1α, 25-dihydroxyvitamin D₃ and analogs of vitamin D because rats and humans metabolize these compounds similarly.

Preparation of 1α, 25-dihydroxyvitamin D₃ dosing solutions. 1α, 25-dihydroxyvitamin D₃ was prepared in two different formulations. Formulation A was an aqueous solution containing 30% propylene glycol and 5% ethanol at pH˜7.0. Formulation B was also an aqueous solution further containing 0.4% TWEEN polysorbate 20 [1 mL of solution contains: 4 mg Tween Polysorbate 20, 1.5 mg sodium chloride, 10 mg sodium ascorbate, 7.6 mg sodium phosphate (dibasic), 1.8 mg sodium phosphate (monodibasic) and 1.1 mg disodium edatate], pH˜7.0.

Intratracheal delivery of 1α,25-dihydroxyvitamin D₃. 5 μCi of 1α,25-dihydroxyvitamin D₃ was delivered intratracheally to anesthetized (isoflurane) Sprague Dawley rats by using the MicroSprayer™ Model 1C following the manufacturer's protocol (PennCentury, Philadelphia, Pa). The device included a stainless steel tube measuring 0.64 mm in diameter attached to a high-pressure syringe (Model FMJ-250, PennCentury). An atomizer at the very tip of the tube generated the aerosol plume. The MicroSprayer™ was inserted deep into the trachea allowing delivery of aerosolized compounds into the lungs. 1α,25-dihydroxyvitamin D₃ was administered in either 100 μL (Formulation A and Formulation B) or 200 μL (Formulation A) dose volumes. As a quench control, a group of animals were dosed with either Formulation A or Formulation B.

Tissue analysis. Using a duration of 4 hours and a duration of 10 minutes after administering the dose, the animals were anesthetized with isoflurane and blood was collected from the heart. The trachea, lungs and stomach were removed, dehydrated in alcohol and pulverized. Samples weighing 20-100 mg were placed in 1 mL Solvable (Packard BioScience B. V., The Netherlands) and incubated at 60° C. overnight. Next, 10 mL of Optima Gold (Perkin Elmer, Boston, Mass.) was added to the samples, and radioactivity was measured using a liquid scintillation analyzer (TRI-CARB 2100 TR, Packard). To determine the presence of 1α,25-dihydroxyvitamin D₃ in the blood, 200-300 μL of serum in 10 mL Optima Gold was used for scintillation counting.

Effectiveness of Aerosol Delivery of Vitamin D to Pulmonary Tree. 1α,25-dihydroxyvitamin D₃ effect on serum calcium levels in Brown Norway rats was determined. Delivery of 1α,25-dihydroxyvitamin D₃ (i.e., in vivo activity) was measured to determine the effect of aerosolized 1α,25-dihydroxyvitamin D₃ treatment on serum calcium levels. Formulation A containing 1α,25-dihydroxyvitamin D₃ was intra-tracheally delivered in 200 μL doses on Day 0 (Day 0 was the first day of treatment) and Day 2. Two different doses, 1 μg/kg_{BW} and 10 μg/kg_{BW}, were tested in two independent experiments. Twenty-four hours after the last dose administration (i.e., Day 2), the animals were anesthetized with isoflurane and blood was collected by heart puncture. The blood was allowed to coagulate at room temperature for at least 30 minutes. The blood was centrifuged at 3000×g (granulated base) for 15 minutes, and the supernatant (serum) was collected. Calcium levels were determined by atomic absorption spectroscopy of the serum diluted 0.1% lanthum chloride using a Perkin Elmer Model 3110.

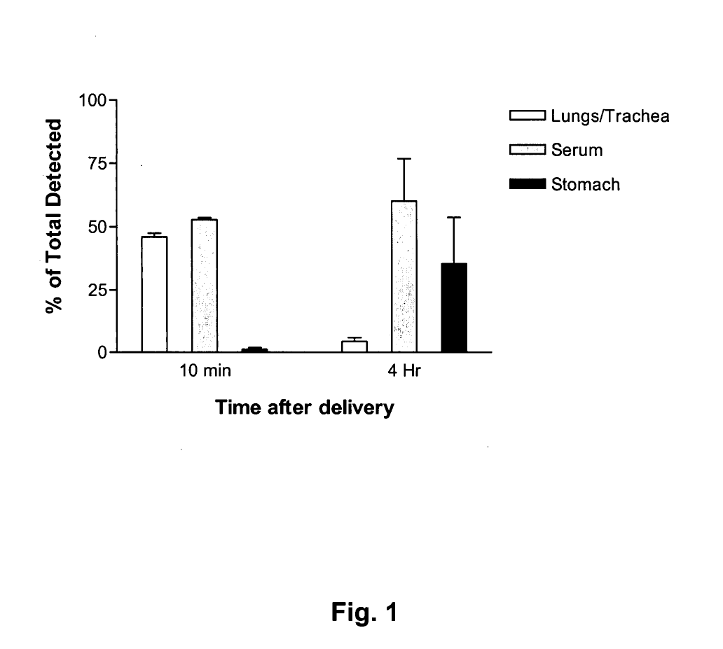

Tissue distribution of 1α,25-dihydroxyvitamin D₃ delivered by Formulation A. As shown in FIG. 1, after 10 minutes, 46% of the detected 1α, 25-dihydroxyvitamin D₃ (100 μL dose) was found in the trachea and lungs, while 53% was detected in the serum. The amount of 1α,25-dihydroxyvitamin D₃ detected in the stomach was very low (˜1%). Similar results were obtained when 1α,25-dihydroxyvitamin D₃ was delivered by a 200 μL dose of Formulation A. After 4 hours, most of the detected 1α, 25-dihydroxyvitamin D₃ was found in the serum (60%) and stomach (36%). Less than 5% was detected in the trachea and lungs.

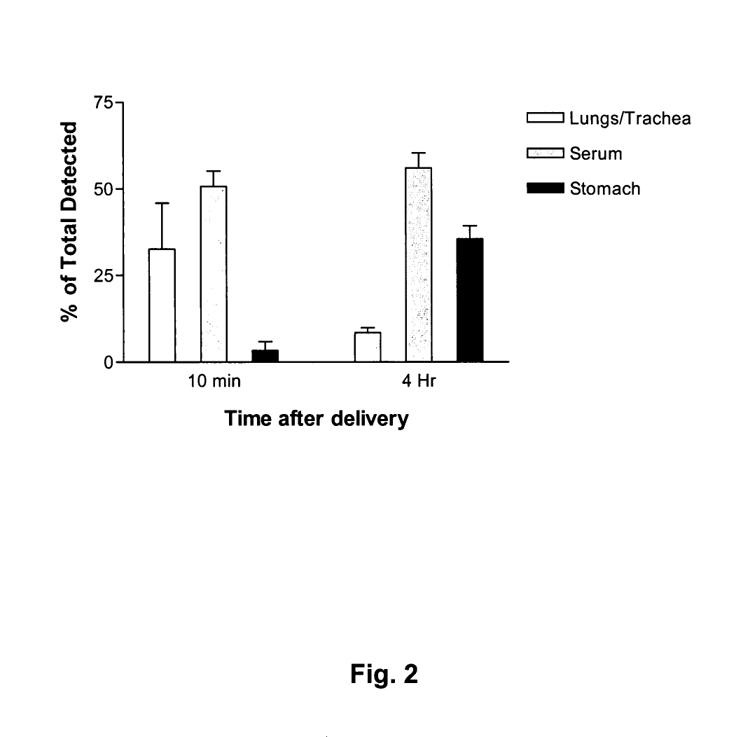

Tissue distribution of 1α,25-dihydroxyvitamin D₃ delivered in Formulation B. As shown in FIG. 2, after 10 minutes, 51% of the detected 1α,25-dihydroxyvitamin D₃ was found in the serum. The trachea and lungs contained 33% of the detected 1α,25-dihydroxyvitamin D₃, and less than 4% was detected in the stomach. After 4 hours, 1α,25-dihydroxyvitamin D₃ was detected in serum (56% of total) and stomach (35% of total).

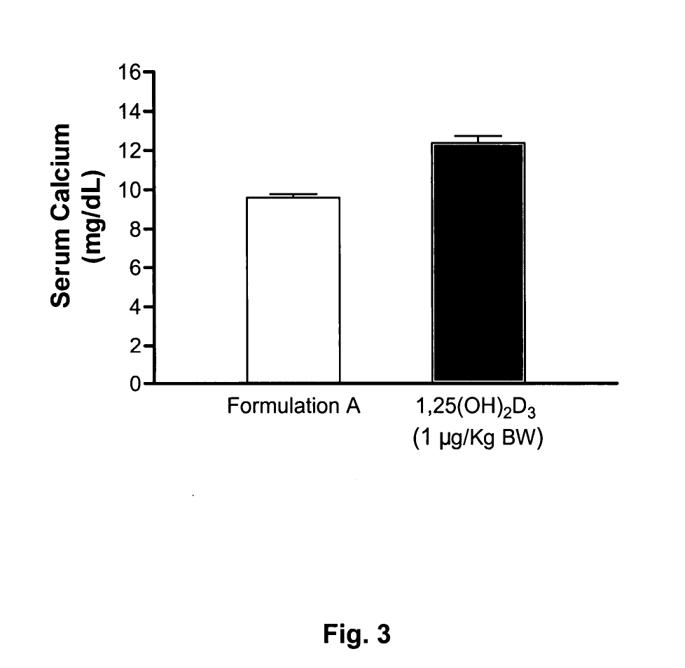

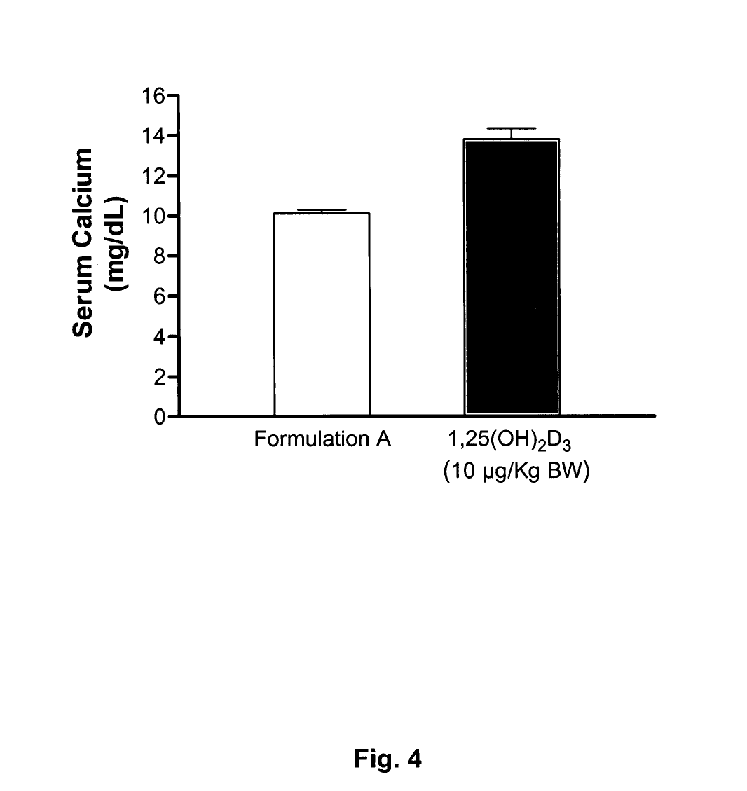

Effect of aerosolized/atomized 1α,25-dihydroxyvitamin D₃ on serum calcium levels. As shown in FIGS. 3 and 4, 1 μg/kg_{BW} and 10 μg/kg_{BW} of pulmonary delivered 1α,25-dihydroxyvitamin D₃ increased serum calcium levels by 2.8 mg/dL and 4 mg/dL, respectively.

The foregoing data demonstrate that 1α,25-dihydroxyvitamin D₃ contained in Formulations A and B were successfully delivered systemically through the pulmonary system. No significant or material difference in tissue distribution was observed by administering Formulations A or B. The presence of 1α,25-dihydroxyvitamin D₃ in the blood 10 minutes after pulmonary delivery indicates that 1α,25-dihydroxyvitamin D₃ was rapidly available. Detection of 1α,25-dihydroxyvitamin D₃ in the stomach 4 hours after dosing indicates that some of the compound probably refluxed into the esophagus. The foregoing examples demonstrate that 1α,25-dihydroxyvitamin D₃ delivered to the pulmonary system efficaciously increased serum calcium levels in animal subjects.

We claim:

1. A method of increasing serum calcium in a human comprising delivering an atomized dose of a pharmaceutical solution comprising water, one or more alcohols, one or more polyols, and, a first active pharmaceutical ingredient comprising 1α,25-dihydroxyvitamin D₃, or esters or solutes thereof to the lungs of the human.

2. The method of claim 1, wherein the alcohol is ethanol.

3. The method of claim 2, wherein the polyol is propylene glycol.

4. The method of claim 3, wherein the pharmaceutical solution further comprises one or more excipients.

5. The method of claim 1, wherein the excipient is a member selected from the group consisting of a nonionic surfactant, sodium chloride, sodium ascorbate, dibasic sodium phosphate, monobasic sodium phosphate, disodium edatate and combinations thereof.

6. The method of claim 1, wherein the atomized dose of the pharmaceutical solution comprises a dose of the first active pharmaceutical ingredient being 1α,25-dihydroxyvitamin D₃ or esters or solutes thereof in the range of 0.2-10 μg.

7. The method of claim 6, wherein the solution further comprises a second active pharmaceutical ingredient selected from the group consisting of calcitonin and N-terminal peptide fragment of parathyroid hormone consisting of the first 34 to 38 amino acids of SEQ ID No. 1.

8. The method of claim 7, wherein the atomized dose of the pharmaceutical solution comprises a dose in the range of 100-2000 μg of the second active pharmaceutical ingredient being the N-terminal peptide fragment of parathyroid hormone consisting of the first 34 to 38 amino acids of SEQ ID No. 1.

9. The method of claim 7, wherein the atomized dose of the pharmaceutical solution comprises a dose in the range of 100-1000 μg of the second active pharmaceutical ingredient being the calcitonin.

10. A pharmaceutical pulmonary composition comprising:
water,
one or more alcohols,
one or more polyols, and,
a first active pharmaceutical ingredient comprising 1α,25-dihydroxyvitamin D₃ or esters or solutes thereof.

11. The composition of claim 10, wherein the alcohol is ethanol.

12. The composition of claim 10, wherein the polyol is propylene glycol.

13. The composition of claim 10, further comprising one or more excipients.

14. The composition of claim 13, wherein the excipient is a member selected from the group consisting of a nonionic surfactant, sodium chloride, sodium ascorbate, dibasic sodium phosphate, monobasic sodium phosphate, disodium edatate and combinations thereof.

15. The composition of claim 10, comprising a dose of the first active pharmaceutical ingredient being 1α,25-dihydroxyvitamin D₃ or esters or solutes thereof in the range of 0.2-10 μg.

16. The composition of claim 10, further comprising a second active pharmaceutical ingredient selected from the group consisting of calcitonin and an N-terminal peptide fragment of parathyroid hormone consisting of the first 34 to 38 amino acids of SEQ ID No. 1.

17. The composition of claim 16, comprising a dose of the second active pharmaceutical ingredient being the N-terminal peptide fragment of parathyroid hormone consisting of the first 34 to 38 amino acids of SEQ ID No. 1 in the range of 100-2000 μg/dose.

18. The composition of claim 16, comprising a dose of the second active pharmaceutical ingredient being the calcitonin in the range of 100-1000 μg/dose.

19. A method of increasing serum calcium in a human comprising delivering a pharmaceutical dose of a first active pharmaceutical ingredient comprising 1α,25-dihydroxyvitamin D₃, or esters or solutes thereof to the lungs of the human.

20. A method of managing hypocalcemia in a human undergoing chronic hemodialysis comprising delivering a pharmaceutical dose of a first pharmaceutical active ingredient comprising 1α,25-dihydroxyvitamin D₃, or esters or solutes thereof to the lungs of the human.

21. A method of treating calcium metabolic disorder in a human comprising delivering a pharmaceutical dose of a first active pharmaceutical ingredient comprising 1α,25-dihydroxyvitamin D₃, or esters or solutes thereof to the lungs of the human.

22. A method of reducing elevated parathyroid hormone levels in a human comprising delivering a pharmaceutical dose of a first active pharmaceutical ingredient comprising 1α,25-dihydroxyvitamin D₃, or esters or solutes thereof to the lungs of the human.

23. The method of any one of claims 19-22, further comprising co-delivering a pharmaceutical dose of a second active pharmaceutical ingredient selected from the group consisting of calcitonin and an N-terminal peptide fragment of parathyroid hormone consisting of the first 34 to 38 amino acids of SEQ ID No. 1.

24. The method of claim 23, comprising a dose in the range of 100-2000 μg of the second active pharmaceutical ingredient being the N-terminal peptide fragment of parathyroid hormone consisting of the first 34 to 38 amino acids of SEQ ID No. 1.

25. The method of claim 23, comprising a dose in the range of 100-1000 μg of the second active pharmaceutical ingredient being the calcitonin.

26. The method of claim 23, comprising a dose of the first active pharmaceutical ingredient being 1α,25-dihydroxyvitamin D₃ or esters or solutes thereof in the range of 0.2-10 μg.

27. A method of increasing serum calcium in a human comprising delivering a dose of a pharmaceutical dry powder composition comprising dry bulking powder and a first active pharmaceutical ingredient comprising 1α,25-dihydroxyvitamin D₃, or esters or solutes thereof to the lungs of the human.

28. A pharmaceutical pulmonary composition comprising:
a dry bulking powder, and,
a first active pharmaceutical ingredient comprising 1α,25-dihydroxyvitamin D₃, or esters or solutes thereof.

29. A method of increasing serum calcium in a human comprising delivering a dose of a pharmaceutical aerosol composition comprising a propellant and a first active pharmaceutical ingredient comprising 1α,25-dihydroxyvitamin D₃, or esters or solutes thereof to the lungs of the human.

30. A pharmaceutical pulmonary composition comprising:
an aerosol propellant, and,
a first active pharmaceutical ingredient comprising 1α,25-dihydroxyvitamin D₃, or esters or solutes thereof.

31. The pharmaceutical pulmonary composition of claim 30, further comprising a surfactant.

32. A dry powder inhaler containing the pharmaceutical pulmonary composition of claim 28.

33. A metered dose inhaler containing the pharmaceutical pulmonary composition of claims 30 or 31.

34. The method of claim 6, wherein the atomized dose of the pharmaceutical solution comprises a dose of the first active pharmaceutical ingredient being 1α,25-dihydroxyvitamin D₃ or esters or solutes thereof in the range of 0.2-2 μg.

35. The composition of claim 15, comprising a dose of the first active pharmaceutical ingredient being 1α,25-dihydroxyvitamin D₃ or esters or solutes thereof in the range of 0.2-2 μg.

36. The method of claim 26, comprising a dose of the first active pharmaceutical ingredient being 1α,25-dihydroxyvitamin D₃ or esters or solutes thereof in the range of 0.2-2 μg.
